(19) United States
(12) Patent Application Publication
(10) Pub. No.: US 2023/0139327 A1
(43) Pub. Date: May 4, 2023

(54) TEST DEVICE

(71) Applicant: SPD Swiss Precision Diagnostics GMBH, Geneva (CH)

(72) Inventors: James Gani, Bedford (GB); Paul James Laverack, Bedfordshire (GB); Sunil Kalbandi, Bedford (GB)

(21) Appl. No.: 17/938,186

(22) Filed: Oct. 5, 2022

Related U.S. Application Data
(63) Continuation of application No. PCT/EP2021/059061, filed on Apr. 7, 2021.

(30) Foreign Application Priority Data
Apr. 7, 2020 (GB) 2005103.3

(51) Int. Cl. B01L 3/00; B01L 3/02

(52) U.S. Cl. CPC B01L 3/502707; B01L 3/021; B01L 2300/0816; B01L 2300/0858; B01L 2200/0642; B01L 2200/10

(57) ABSTRACT

The present invention relates to test devices comprising one or more retaining members for removably retaining a sample applicator, sample applicators for use with such test devices, and kits comprising test devices and sample applicators of the invention.

### CROSS-REFERENCE TO RELATED APPLICATIONS

This application is a Continuation of International Application No. PCT/EP2021/059061, filed on Apr. 7, 2021, which claims the benefit of UK Pat. App. No. GB 2005103.3, filed on Apr. 7, 2020. The entirety of each of these applications is incorporated herein by reference in their entirety.

### FIELD

The present invention relates to test devices comprising one or more retaining members for removably retaining a sample applicator, sample applicators for use with such test devices, and kits comprising test devices and sample applicators of the invention.

### BACKGROUND

Various diagnostic products are known which analyse a fluid sample, such as urine or blood, to determine the presence or amount of one or more analytes. These may be small, handheld devices, which are used by applying a biological sample to an absorbent component. The devices are configured subsequently to convey the fluid along a flow path to an assay test zone, where a reaction or binding event takes place. Typically, such devices include a window through which the test zone is visible, such that an indication of the assay result can be observed by a user.

Such test devices may be provided with or packaged alongside a sample applicator, such as a pipette, for applying sample to the device. In some known devices, a sample applicator is packaged separately to the test device or is retained on the side of the device. There are various problems with such arrangements. For example, retaining an applicator on the side of a test device increases the overall footprint of the product, making it undesirably bulky and resulting in the need for more packaging material. A further problem is that the presence of the sample applicator can hinder efficient packaging processes, such as flow wrapping, by getting caught on the packaging material during the packaging process.

### SUMMARY

According to an aspect, the invention provides a test device comprising a housing and a test element, the test element comprising, a sample application zone, an analyte detection zone, a sample flow path, the test device further comprising at least one retaining member for removably retaining a sample applicator in a retained position, wherein
a. at least one retaining member does not substantially extend beyond one or more boundaries of the housing of the test device, and/or
b. wherein when the sample applicator is in the retained position, the sample applicator does not extend substantially beyond one or more boundaries of the housing of the test device.

The test device comprises a test element comprising a sample application zone, an analyte detection zone and a sample flow path. Preferably, the test element is either partially or completely contained within the housing. The test element may be a lateral flow assay strip. The lateral flow assay strip may be of the sort disclosed in EP291194 or U.S. Pat. No. 6,352,862. In operation, sample is applied to the sample application zone and flows along the sample flow path to the analyte detection zone where an analyte of interest can be detected. In some instances, the sample flow path may further comprise mobilizable labelled reagents downstream of the sample application zone. The analyte detection zone typically comprises a test zone or test line and may also comprise a control zone or control line. The result of the assay may be viewable at the analyte detection zone, for example in the form of a coloured line which forms at the test zone in the presence of analyte. The control zone is typically positioned downstream of the test zone and functions to show that sample has been applied and that the mobilizable labelled reagents have functioned correctly i.e. they are mobile and functional.

The test device comprises a housing. The housing of a test device typically defines the boundary of the device and protects the internal components of the device, such as the test element. The housing may serve additional functions, such as, for example, providing instructions to the user. The housing typically comprises a base, on which the device may sit if placed on a surface or held in a user's hand, and a top surface on the side of the device opposing the base. The top surface of the housing may comprise cut-outs and/or windows which allow the user to apply sample to the device and facilitate viewing of the assay result, such as an analyte detection zone window. The top surface may comprise a sample application well for applying sample to the device. The well is typically in liquid communication with the sample application zone of the test element. The housing also typically further comprises a side wall which may join the base and top surface and extend in a substantially perpendicular plane to the base and the top surface. The device may rest on the base, side wall or a combination of the base and side wall, when placed on a horizontal surface. The test device may comprise a base, a top surface and a side wall substantially perpendicular to the base and/or the top surface. The test device may comprise a retaining member in the side wall and/or in the top surface. For example, the test device may comprise a retaining member, for example a recess, which spans a portion of the side wall and a portion of the top surface. Boundaries of the housing may include:

a boundary of the device housing defined by a first plane extending in a vertical direction at a proximal end of the device located along the horizontal y axis of the device;

a boundary of the device housing defined by a second plane extending in a vertical direction at a distal end of the device located along the horizontal y axis of the device;

a boundary of the device housing defined by a third plane extending in the vertical direction at a proximal end of the device located along a horizontal x axis of the device;

a boundary of the device housing defined by a fourth plane extending in the vertical direction at a distal end of the device located along a horizontal x axis of the device;

a boundary of the device housing defined by a fifth plane extending in a horizontal direction from the uppermost point of the housing.

The side wall may extend above the top surface of the device at one or more locations around the perimeter of the device. The side wall may extend above the top surface around the entire perimeter or substantially the entire perimeter of the device. The distance by which the side wall extends above the top surface may or may not be substantially uniform around the perimeter of the device. For example, in some embodiments, the side wall may extend above the top surface to a greater extent in some regions of the perimeter than in others. Test devices, and hence test device housings, typically extend in the horizontal plane along one axis further than the other (particularly where the device comprises a lateral flow assay strip) and therefore typically have a longer (y) axis and a shorter (x) axis. Test devices and test device housings also have a vertical (z) axis.

The test device further comprises one or more retaining members for removably retaining a sample applicator. In a preferred embodiment, at least one, and preferably all retaining members do not extend substantially (or at all) beyond one or more boundaries of the housing of the test device, as described herein. In an embodiment, at least one, and preferably all retaining members do not extend substantially (or at all) beyond any boundary of the housing of the test device. In an embodiment, at least one, and preferably all retaining members do not extend substantially (or at all) beyond any combination of boundaries (i)-(v), such as for example, boundaries (i)-(iv), boundaries (i) and (ii), boundaries (iii) and (iv), etc as described herein.

The test device may comprise retaining members at opposing ends of the housing. If multiple retaining members are used, preferably none of the retaining members extends substantially (or at all) beyond the housing of the test device. For example, in some embodiments, the or each retaining member does not extend substantially (or at all) beyond a side wall of the housing. This has the advantage that the overall footprint of the device is minimised and the device is more streamlined, meaning that it can be packaged more efficiently. Additionally, a retaining member which does substantially extend from the housing of the test device may be vulnerable to damage during packaging and transportation (for example, it could snap off), and there is also a risk that that such a retaining member could penetrate the packaging. If the packaging is penetrated the test device will no longer be held in a desiccated state, which is necessary to preserve the integrity of the reagents and hence their functionality. The present invention advantageously does not suffer from such problems.

In another embodiment, when the sample applicator is in the retained position, the sample applicator does not extend substantially (or at all) beyond one or more boundaries of the housing of the device. In an embodiment, when the sample applicator is in the retained position, the sample applicator does not extend substantially (or at all) beyond any boundaries of the housing of the device. In an embodiment, in the retained position, the sample applicator does not extend substantially (or at all) beyond any combination of boundaries (i)-(v), such as for example, boundaries (i)-(iv), boundaries (i) and (ii), boundaries (iii) and (iv), etc as described herein.

In an embodiment, the horizontal y axis is longer than the horizontal x axis of the device.

In an embodiment, a retaining member for retaining a sample applicator does not form part of the housing for the purposes of defining a boundary of the housing.

Thus, the sample applicator may be retained in a position such that it does not extend substantially beyond the footprint of the device. The sample applicator may be retained above the top surface of the housing.

By “does not extend substantially” it is meant that at least a majority of the component (such as the sample applicator or a retaining member) does not extend beyond the relevant boundary. In some embodiments, less than 50%, less than 25%, less than 10%, less than 5%, less than 1%, or 0% of the volume of the component extends beyond the relevant boundary.

In an embodiment, when the sample applicator is in a retained position, less than 50%, less than 25%, less than 10%, less than 5%, or less than 1%, or 0% of the volume of the sample applicator extends beyond one or more boundaries of the housing of the test device.

In an embodiment, less than 50%, less than 25%, less than 10%, less than 5%, or less than 1%, or 0% of the volume of a retaining member (preferably all retaining members) extends beyond one or more boundaries of the housing of the test device.

The retaining member may comprise a recess configured to retain an end, for example a tip, of the sample applicator. The recess may be provided in the housing of the test device. The recess may be provided in a side wall of the housing. The recess may be provided in a top surface of the housing. In a preferred embodiment, the recess is provided in a portion of the side wall which extends above the top surface of the housing. In this instance the recess may be configured such that an end, for example a tip, of the sample applicator can be inserted into the recess. The recess may partially or completely surround a portion of an end of the sample applicator.

Alternatively, or additionally, a retaining member may comprise a recess configured to retain a retaining lug of the sample applicator. The recess may be provided in the housing of the test device. The recess may be provided in a side wall of the housing. The recess may be provided in a top surface of the housing. In a preferred embodiment, the recess is provided in a portion of the side wall which extends above the top surface of the housing. The recess may be generally “U” shaped, so that the retaining lug of the sample applicator can be pushed into the recess from above. Variations on this approach will be apparent to the skilled person. The retaining lug and the recess are preferably tight fitting such that the retaining lug is held in the recess by friction after the parts are pushed together. This is known as “press fit” or “interference fit”. In a preferred embodiment, a retaining member and sample applicator engage by press fit. Where the test device has a relatively longer horizontal axis, a relatively shorter horizontal axis and a vertical (z) axis, the press fit may be operated in the direction of the vertical (z) axis. The relatively longer horizontal axis may be referred to as the y axis and the relatively shorter horizontal axis may be referred to as the x axis, or vice versa.

Alternatively, or additionally, a retaining member may comprise a recess, for example a cup-shaped recess, configured to retain a bulb (or other portion) of the sample applicator. The recess may be provided in the housing of the test device. The recess may be provided in a side wall of the housing. The recess may be provided in a top surface of the housing. In this embodiment, a portion of the sample applicator may be compressed to reduce its volume allowing it to fit at least partially within the recess. The applicator will then be retained as it expands. A recess may, for example, span a portion of the top surface and the side wall.

The retaining member may comprise both a first recess configured to retain a first end, for example a tip, of the sample applicator and a second recess configured to retain a second end, for example a retaining lug, of the sample applicator. The first and the second retaining recess may be provided at opposing ends of the test device. For example, the first recess may be provided at a first end of a longer (y) horizontal axis of the device, and the second recess may be provided at the opposing end of the longer (y) axis of the device.

A retaining member may comprise one or more projections configured to retain a portion, for example an end, such as a tip and/or retaining lug, of a sample applicator. The retaining member may comprise a projection from a side wall of the device in a portion of the side wall which extends above the top surface of the housing. A projection may cooperate with a hollow tip of the applicator to retain an end of the applicator.

A retaining member may comprise one or more clips configured to retain one or more portions of the sample applicator, such as the tip, barrel, and/or retaining lug of a sample applicator.

A retaining member may comprise a projection and/or a clip.

In use, the test device is placed with the base facing downwards on a horizontal surface or in the hand of a user. This position is referred to as the horizontal test position. When the test device is in a horizontal test position, and the sample applicator is in the retained position, the sample applicator may be located substantially above at least one of the sample flow path, the analyte detection zone, the test zone, the control zone, and the sample application zone. The sample applicator will typically have a longer (y) horizontal axis and shorter (x) horizontal axis. The longer (y) horizontal axis may be aligned with a longer (y) horizontal axis of the test device in the retained position.

When the sample applicator is in a retained position the sample applicator may cover or at least partially obscure the sample application zone and/or the sample application well, where present. When the sample applicator is in a retained position the sample applicator may cover or at least partially obscure the analyte detection zone, the test zone, and/or the control zone.

The sample applicator described herein may be a pipette, for example, a Pasteur pipette. The pipette may comprise a tip, a barrel and a bulb. Preferably, the pipette further comprises a retaining lug which is configured to be retainable by the retaining structure. In use, liquid sample is drawn up through the tip into the barrel of the pipette by the action of squeezing and releasing the bulb. The liquid sample is released from the pipette by the action of squeezing the bulb. A precise and reproducible amount of sample can be released from the pipette.

In a further aspect, the invention provides a test device comprising a housing, a test element, the test element comprising a sample application zone, an analyte detection zone, and a sample flow path, the test device further comprising at least two retaining members for removably retaining a sample applicator in a retained position, wherein the housing comprises a base, a top surface and a side wall substantially perpendicular to the base and/or the top surface, wherein
a. a first retaining member comprises a first recess in the side wall, the recess being configured to retain a first end of the sample applicator, and
b. a second retaining member comprises a second recess in the side wall, the second recess being configured to retain a second end of the sample applicator.

The first retaining member and the second retaining member may be at opposing ends of the test device. The first end of the sample applicator may comprise a tip of the sample applicator and the first recess may retain the tip. The second end of the sample applicator may comprise a retaining lug and the second recess may retain the retaining lug.

According to a further aspect, the invention provides a sample applicator a retaining lug configured to engage with a retaining member of a test device to retain the sample applicator. The sample applicator may be in the form of a pipette, comprising a bulb, a barrel and a tip, wherein the bulb comprises a retaining lug. The retaining lug is preferably configured to engage a retaining member of a test device as described herein.

According to another aspect, the invention provides a kit comprising a test device and a sample applicator as described herein.

All features of each aspect of the invention are applicable to the other aspects of the invention unless otherwise indicated.

### BRIEF DESCRIPTION OF THE DRAWINGS

Embodiments of the invention will now be described, by way of example, with reference to the drawings, in which:

FIG. 1 shows a line drawing of an assay device and sample applicator in the retained position according to one embodiment of the invention.

FIG. 2 shows a line drawing of an assay device and sample applicator of FIG. 1 in the unretained position.

### DETAILED DESCRIPTION

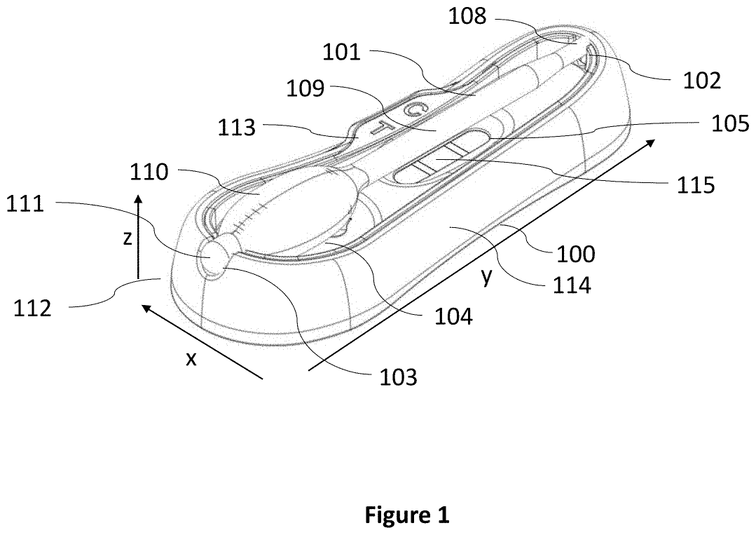

The test device of the invention can be further understood with reference to FIG. 1, which shows a test device (100) comprising a test element located within a housing (112). The housing extends along two horizontal axes, labelled x and y, and a vertical axis, labelled z. The housing extends further along the y axis than the x axis. A removable sample applicator (101) is releasably retained on the test device. The housing (112) comprises a top surface (113), a bottom surface (not visible in the figure) substantially parallel to the top surface and a side wall (114) joining the top and bottom surfaces. The side wall extends generally in the direction of the vertical z axis of the device. The test device has a distal end situated along the y axis comprising a first recess (102), and a proximal end situated along the y axis comprising a second recess (103). The first recess (102) is in a portion of the side wall (114) which extends above the top surface (113) of the housing. The second recess (103) is also in a portion of the side wall which extends above the top surface (113) of the housing (112). The test element (not visible in the figure) contained within the housing (112) comprises an analyte detection zone (115). The analyte detection zone (115) comprises a test zone and a control zone (identified as lines T and C respectively in the figure). The top surface (113) comprises an analyte detection zone window (105) for viewing the analyte detection zone (115). A sample application well (104) is provided in the top surface (113) for applying sample to the sample application zone of the test element using the sample applicator. The removable sample applicator (101) comprises a tip (108), a bulb (110) and a barrel (109) connecting the tip (108) to the bulb (110). A retaining lug (111) is provided at the end of the bulb (110) that does not connect to the barrel (109).

The test device is configured such that the tip (108) of the sample applicator (101) is received by the first recess (102), and the retaining lug (111) of the sample applicator (101) is received by the second recess (103). The first recess (102) is configured such that a tip (108) of the sample applicator (101) can be inserted into the first recess (102).

The second recess (103) is “U” shaped, so that the retaining lug (111) of the sample applicator (101) can be pushed into the recess (103) from above. The retaining lug (111) is held in the second recess (103) by friction after the parts are pushed together i.e. by a press fit. Advantageously, when the sample applicator is in the retained position, neither the retaining members nor the sample applicator extend substantially beyond the housing of the test device. Furthermore, in this embodiment, the tip is tucked inside the side wall of the housing. This minimises the likelihood that the sample applicator will be caught on packaging or otherwise interfere with the packaging process. It also minimises the amount of packaging required.

In the retained position shown in FIG. 1, the sample applicator at least partially obscures the sample application well (104), sample application zone, and the analyte detection zone window (105). This is advantageous in that it indicates to the user that, in order to commence an assay, the sample applicator must first be removed from the test device.

In order to use the sample applicator to apply liquid to the test device, the sample applicator is removed from the test device by lifting it upwards so that the retaining lug (111) disengages the second recess (103) and the tip (108) is withdrawn from the first recess (102).

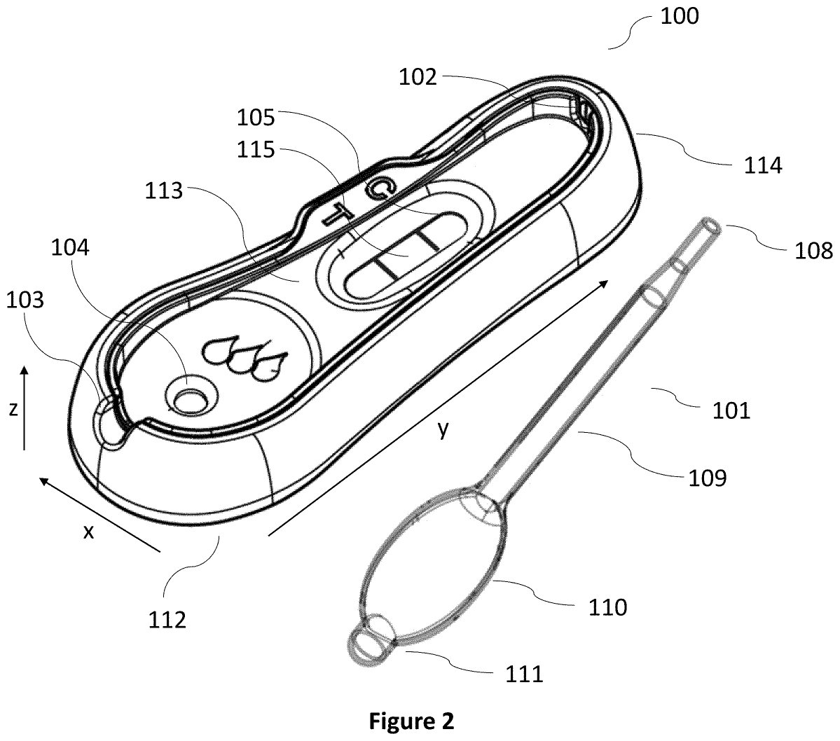

FIG. 2 shows the test device and sample applicator of FIG. 1 separately i.e. in the unretained state. The sample well (104) is no longer obscured by the sample applicator (101) and is accessible allowing a user to apply sample to the test device (100) using the sample applicator (101). Similarly, the analyte detection zone window (105) is no longer obscured by the sample applicator (101) and can be readily observed by a user.

1. A test device comprising a housing and a test element, the test element comprising a sample application zone, an analyte detection zone, and a sample flow path, the test device further comprising at least one retaining member for removably retaining a sample applicator in a retained position, wherein
at least one retaining member does not extend substantially beyond the housing of the test device, and/or
wherein when the sample applicator is in the retained position, the sample applicator does not extend substantially beyond one or more boundaries of the housing of the test device.

2. The test device according to claim 1, comprising retaining members at opposing ends of the housing.

3. The test device according to claim 1, wherein the at least one retaining member comprises;
a) a first recess configured to retain an end, such as a tip, of the sample applicator, and/or
b) a second recess configured to retain a retaining lug of the sample applicator.

4. The test device according to claim 3, wherein the at least one retaining member comprises the first recess and the second recess.

5. The test device according to claim 1, wherein the housing comprises a base, a top surface and a side wall substantially perpendicular to the base and/or the top surface.

6. The test device according to claim 5, wherein the at least one retaining member is in the side wall and/or top surface.

7. The test device according to claim 6, wherein the at least one retaining member and the sample applicator engage by press fit.

8. The test device according to claim 1, wherein the test device has a horizontal (y) axis, a horizontal (x) axis shorter than the horizontal (y) axis, and a vertical (z) axis and wherein the press fit is operated in the direction of the vertical (z) axis.

9. The test device according to claim 1, wherein in the retained position, the sample applicator does not extend substantially, or at all, beyond one or more of the following:
i. a boundary of the device housing defined by a first plane extending in a vertical direction at a proximal end of the device located along the horizontal y axis of the device;
ii. a boundary of the device housing defined by a second plane extending in a vertical direction at a distal end of the device located along the horizontal y axis of the device;
iii. a boundary of the device housing defined by a third plane extending in the vertical direction at a proximal end of the device located along a horizontal x axis of the device;
iv. a boundary of the device housing defined by a fourth plane extending in the vertical direction at a distal end of the device located along a horizontal x axis of the device;
v. a boundary of the device housing defined by a fifth plane extending in a horizontal direction from the uppermost point of the housing.

10. The test device according to claim 1, wherein the at least one retaining member comprises a projection and/or a clip.

11. The test device according to claim 1, wherein when the test device is in a horizontal test position, and the sample applicator is in the retained position, the sample applicator is located substantially above at least one of the sample flow path, the analyte detection zone, and the sample application zone.

12. The test device according to claim 1, wherein
a) when the sample applicator is in a retained position, less than 50%, less than 25%, less than 10%, less than 5%, or less than 1%, or 0% of the volume of the sample applicator extends beyond one or more boundaries of the housing of the test device, and/or
b) less than 50%, less than 25%, less than 10%, less than 5%, or less than 1%, or 0% of the volume of the retaining member extends beyond one or more boundaries of the housing of the test device.

13. The test device according to claim 1, wherein
(a) when the sample applicator is in a retained position, the sample applicator at least partially obscures the sample application zone and/or the analyte detection zone, and/or
(b) the test device comprises a sample application well in communication with the sample application zone, and an analyte detection zone window, and when the sample applicator is in a retained position the sample applicator at least partially obscures the sample application well and/or the analyte detection zone window.

14. A test device comprising a housing, and a test element, the test element comprising a sample application zone, an analyte detection zone, and a sample flow path, the test device further comprising at least two retaining members for removably retaining a sample applicator in a retained position, wherein the housing comprises a base, a top surface and a side wall substantially perpendicular to the base and/or the top surface, wherein
a first retaining member comprises a first recess in the side wall and/or top surface, the recess being configured to retain a first end of the sample applicator, and
a second retaining member comprises a second recess in the side wall and/or top surface, the second recess being configured to retain a second end of the sample applicator.

15. The test device according to claim 14, wherein the first retaining member and the second retaining member are at opposing ends of the test device.

16. The test device of claim 14, where the first end of the sample applicator comprises a tip of the sample applicator and the first recess retains the tip and/or wherein the second end of the sample applicator comprises a retaining lug and the second recess retains the retaining lug.

17. A sample applicator comprising a retaining lug configured to engage with a retaining member of a test device to retain the sample applicator.

18. The sample applicator according to claim 17, wherein the applicator is a pipette comprising a tip, a barrel, a bulb, wherein the bulb comprises the retaining lug.

19. The sample applicator of claim 18 wherein the test device comprises a housing, and a test element, the test element comprising a sample application zone, an analyte detection zone, and a sample flow path, the test device further comprising at least two retaining members for removably retaining a sample applicator in a retained position, wherein the housing comprises a base, a top surface and a side wall substantially perpendicular to the base and/or the top surface, wherein
a first retaining member comprises a first recess in the side wall and/or top surface, the recess being configured to retain a first end of the sample applicator, and
a second retaining member comprises a second recess in the side wall and/or top surface, the second recess being configured to retain a second end of the sample applicator.

20. A kit comprising a test device according to claim 16, wherein the sample applicator is a pipette, the pipette comprising a tip, a barrel, a bulb, and wherein the bulb comprises the retaining lug.

21. The sample applicator of claim 18, wherein the test device comprises:
a housing and a test element, the test element comprising a sample application zone, an analyte detection zone, and a sample flow path,
the test device further comprising at least one retaining member for removably retaining a sample applicator in a retained position, wherein
at least one retaining member does not extend substantially beyond the housing of the test device, and/or
wherein when the sample applicator is in the retained position, the sample applicator does not extend substantially beyond one or more boundaries of the housing of the test device.

22. A kit comprising a test device according to claim 3, wherein the sample applicator is a pipette, the pipette comprising a tip, a barrel, a bulb, and wherein the bulb comprises the retaining lug.
